(19) United States
(12) Patent Application Publication
(10) Pub. No.: US 2010/0076302 A1
(43) Pub. Date: Mar. 25, 2010

(54) Invisible Power Port ID Tattoo

(76) Inventors: Jeff Gray, Lexington, MA (US); Stephanie Dubay, Westford, MA (US); Paul DiCarlo, Middleboro, MA (US)

Correspondence Address: Bingham McCutchen LLP, 2020 K Street, NW, Washington, DC, 20006, US

(21) Appl. No.: 12/238,094

(22) Filed: Sep. 25, 2008

(51) Int. Cl. A61B 6/12; A61B 8/00

(52) U.S. Cl. 600/424; 600/431

(57) ABSTRACT

A solution for use in medical procedures, comprises including a plurality of microspheres holding a fluorescent dye reactive to ultraviolet light and a base dye which, under white light, has one of a color substantially matching a color of a target portion of skin into which it is to be injected and which has, under UV light, a color slightly darker than a color of the target portion of skin. A method comprises identifying a target area of the skin to be accessed in a medical procedure and injecting into at least a portion of the target area a solution including a plurality of microspheres holding a colored dye and a fluorescent dye reactive to ultraviolet light in combination with locating the target area by illuminating the skin with ultraviolet light.

### BACKGROUND

Subcutaneously implanted devices are often difficult to locate in the body. Various techniques have been devised to facilitate the location and identification such devices including simple palpation of the skin. However, this can be time consuming and this is often insufficient to permit a clinician to distinguish between multiple implanted devices. Thus, when multiple devices have been implanted, a patient may be required to carry an identification item (e.g., a wallet card, ID bracelet key chain, etc.) indicating the shape and function of each implanted device. If such identification is unavailable or if an implanted device is otherwise unidentifiable, an intermediate step of a CT scan or an x-ray may be required to positively identify the device, increasing the cost and difficulty of the procedure.

Furthermore, certain procedures require access to areas of the body for a prolonged period of time, such as in radiation therapy for the treatment of cancer. For such treatments, it is vital that a physician locate a particular cancerous portion of the body and apply radiation therapy only to that particular location. Those skilled in the art will understand the harmful side effects that may be induced by a miscalculation of a radiation therapy treatment location. Accordingly, some physicians use medical tattoos to map out radiation therapy treatment regions. These tattoos are visible under ordinary light and thus may be distasteful to many patients. Nevertheless, because of the importance of radiation therapy most patients swallow their reservations and agree to the tattooing.

### SUMMARY OF THE INVENTION

The present invention is directed to a solution for use in medical procedures, comprising a plurality of microspheres or nanospheres holding a fluorescent dye reactive to ultraviolet light and a base dye which, under white light, having one of a color substantially matching a color of a target portion of skin into which it is to be injected and, under ultraviolet light, has a color slightly darker than a color of the target portion of skin.

The present invention is further directed to a method comprising identifying a target area of the skin to be accessed in a medical procedure and injecting into at least a portion of the target area a solution including a plurality of microspheres or nanospheres holding a colored dye and a fluorescent dye reactive to ultraviolet light in combination with locating the target area by illuminating the skin with ultraviolet light.

### BRIEF DESCRIPTION OF THE DRAWINGS

The accompanying drawings illustrate the present invention.

FIG. 1 shows a microsphere according to the present invention encasing a color dye molecule, the microsphere being shown in relation to a red blood cell;

FIG. 2 shows an exemplary design of an ID tattoo according to an embodiment of the present invention;

FIG. 3 shows a view under normal light of a hand tattooed with ink according to the present invention; and

FIG. 4 shows a view of the hand of FIG. 3 under blacklight.

### DETAILED DESCRIPTION

The present invention, which may be further understood with reference to the following description and the appended drawings, relates to a system and method for the identification of an implanted medical device in a body. It is noted that although the exemplary embodiments of the present invention are described below with respect to particular procedures and anatomical locations, the description is not meant to limit the application of the invention, which may be employed in a plurality of body regions for a wide variety of reasons.

A system and method according to an exemplary embodiment of the present invention employs a tattoo which is invisible under ordinary (white) light to facilitate various procedures. The tattoo of the present invention is visible under blacklight to serve as an indicator of the location and/or function of an implanted medical device, a mapping area for radiation therapy, etc. As would be understood by those skilled in the art, a blacklight for use in relation with the present invention may be formed in the same fashion as normal fluorescent lights except that only one phosphor is used and the normally clear glass envelope of the fluorescent bulb may be replaced by Wood's Glass, a nickel-oxide doped glass which blocks all visible light above approximately 400 nanometers. Specifically, the intensity of the tattoo of the present invention may peak in the range of approximately 350-405 nm and, more particularly, in the range of 350-370 nm, wherein the location of the intensity peak is dependent on the type of glass used in the blacklight bulb, as those skilled in the art will understand. Accordingly, at light wavelengths within the optical spectrum but outside of the blacklight peak range, the tattoo of the present invention remains invisible.

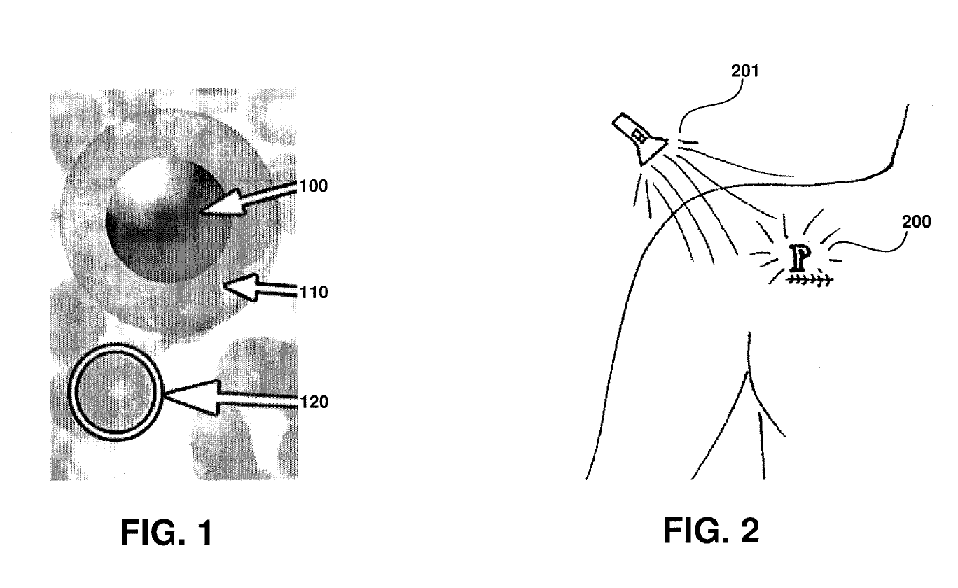

As shown in FIG. 2, a tattoo 200 according to the present invention employs blacklight reactive ink comprising color dye molecules 100 embedded within polymethylmethacrylate (“PMMA”) microspheres 110. In another embodiment of the present invention, the microspheres 110 may be composed of a polymer material. As indicated in FIG. 1, the PMMA microspheres 110 are approximately 4-5 times as large as a human red blood cell 120. Due to its encasement in the PMMA microspheres 110, which essentially serves as a shield for the color dye molecule 100, the color dye molecules 100 of the present invention do not come into direct contact with the skin of a patient. Those skilled in the art will understand that nanospheres may be substituted for the described microspheres without departing from the scope of the invention and, as used herein, the term microspheres encompasses nanospheres as well.

The color dye molecules 100 loaded in the PMMA microspheres 110 comprise a base that is generally a color darker than a skin color of a patient in combination with a fluorescent dye. Alternatively, as discussed in greater detail hereinafter, the base may be substantially the same color as a skin color of a patient. As shown in FIG. 2, the fluorescent dye of the tattoo 200, when viewed under an ultraviolet (“UV”) blacklight 201, exhibits an intense fluorescence due to phosphors within the dye which convert energy from the UV radiation into visible light. The combination of the two components in the color dye molecules 100 results in a colored tattoo that is only slightly darker than the patients's skin and is only visible when exposed to blacklight from, for example, a UV blacklight 201.

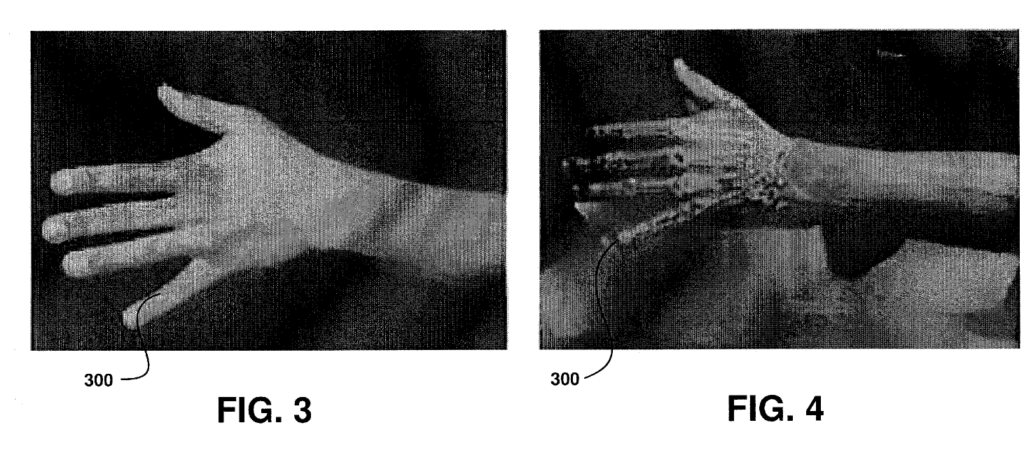

As shown in FIG. 3, a tattoo 300 according to an embodiment of the invention is invisible under normal lighting conditions while under blacklight, as shown in FIG. 4, the highlighted portions of the skin clearly show the pattern of the tattoo 300.

According to a method of use of the tattoo ink of the present invention, a tattoo is applied after a target area of the skin has been identified (e.g., following a medical device implantation procedure, an x-ray procedure indicating the location of a target site within the body such as a tumor, etc.). A syringe (e.g., a single-use syringe) may then be filled with ink according to the invention or, alternatively, may be purchased pre-loaded with this ink (e.g., in a port kit including a single use safety needle). The injection process itself may vary on a case to case basis. For example, when used to indicate the location of a port in the body, the injection process may comprise the injection of the tattoo ink on a location atop a central axis of the implanted device in a pattern which optionally indicates a type of the device (e.g., whether a port is suitable or unsuitable for power injection). When used to indicate mapping for cancer radiation therapy, the injection process may comprise a series of injections that may form a substantially solid line spanning the length of the required treatment area, wherein the thickness of the substantially solid line may vary according to the thickness of the required treatment area. Alternatively, any injection pattern may be employed such as, for example, a pattern matching the size and/or anatomy of the target site within the body. For example, a user may make multiple punctures to inject individual dots of the ink at predetermined marginal distances wherein the need for multiple punctures may correlate to the size and anatomy of the target site within the body. The tattoo of the present invention is preferably injected into a dermis of the skin with a needle size and depth known to those skilled in the art. In one embodiment, the tattoo may be injected to approximately 4.25-5 mm below the skin.

In another embodiment of the present invention, the tattoo ink may blend unobtrusively with the patient's own skin color, as opposed to exhibiting a slightly darker shade as noted above in the embodiment of FIGS. 1-4. This embodiment permits a tattoo that is virtually unnoticeable under normal conditions (i.e., in the absence of UV blacklight).

In yet another embodiment of the present invention, the tattoo may incorporate a filler that is reactive to ultrasonic waves. The filler may be used along with the dye molecules 100 in the microsphere 110 of FIG. 1 or, alternatively, may be used in place of the dye molecules 100. This embodiment may allow a clinician to view the tattoo by passing a wand of an ultrasound machine over the tattooed portion of skin, as those skilled in the art will understand. The filler material may comprise microspheres or nanospheres with air bubble cores so that the air bubble core will resonate with the ultrasonic energy. Alternatively, the microspheres may comprise another gas filler such as, but not limited to, a perfluorocarbon compound, nitrogen, xenon, argon, helium, nitrous oxide, carbon dioxide.

In yet another embodiment of the present invention, the PMMA microsphere 110 may be filled with standard tattoo dye in addition to the fluorescent dye so that all or portions of the tattoo may be visible under white light and UV blacklight.

Those skilled in the art will understand that the described exemplary embodiments of the present invention may be altered without departing from the spirit or scope of the invention. Thus, it is to be understood that these embodiments have been described in an exemplary manner and are not intended to limit the scope of the invention which is intended to cover all modifications and variations of this invention that come within the scope of the appended claims and their equivalents. The specifications are, therefore, to be regarded in an illustrative rather than a restrictive sense.

What is claimed is:

1. A solution for use in medical procedures comprising a plurality of microspheres, the microspheres holding a first dye reactive to ultraviolet light and a second dye which, under white light has one of a color substantially matching a color of a target portion of a patient's skin into which it is to be injected and, under ultraviolet light, has a color different than a color of the target portion of skin.

2. The solution according to claim 1, wherein at least a portion of the microspheres comprise polymethylmethacrylate.

3. The solution according to claim 1, wherein the first dye is encapsulated in a fluid tight polymer sealed to fluid flow, the encapsulated first dye being suspended in the second dye.

4. The solution according to claim 1, wherein the microspheres are formed of a material non-reactive with the skin.

5. The solution according to claim 1, wherein at least a portion of the microspheres include a filler of one of air, a perfluorocarbon gas and nitrogen.

6. The solution according to claim 5, wherein the filler material is reactive to ultraviolet light.

7. The solution according to claim 5, wherein the filler material is reactive to ultrasonic waves.

8. The solution according to claim 1, wherein the dye is separated from red blood cells of the target portion of skin by a wall of the microsphere.

9. The solution according to claim 1, wherein the second dye is darker than the target portion of the patient's skin.

10. A method, comprising:
identifying a target area of a patient's skin to be accessed in a medical procedure;
injecting into at least a portion of the target area a solution including a plurality of microspheres, the microspheres comprising a phosphorescent dye, reactive under a predetermined wavelength of ultraviolet light, encapsulated by a material which does not absorb ultraviolet light; and
locating the target area by illuminating the skin with ultraviolet light.

11. The method of claim 10, wherein the target area is a subcutaneous region of interest.

12. The method of claim 11, wherein the subcutaneous region of interest includes one or both of an implanted device and a subcutaneous portion of tissue to be treated.

13. The method of claim 10, further comprising inserting a therapeutic device into the target area to perform a therapeutic procedure.

14. A method, comprising:
identifying a target area of the skin to be accessed in a medical procedure;
injecting into at least a portion of the target area a solution including a plurality of microspheres holding a gas reactive to ultrasonic waves; and
locating the target area by passing ultrasound energy through the skin until a pattern indicative of the presence of the gas containing microspheres is identified.

15. The method of claim 14, wherein the gas is one of air, a perfluorocarbon gas and nitrogen.

16. The method of claim 14, wherein the target area is a subcutaneous region of interest.

17. The method of claim 16, wherein the subcutaneous region of interest includes one of an implanted device and a portion of subcutaneous tissue to be treated.
